(19) United States
(12) Patent Application Publication
(10) Pub. No.: US 2026/0097128 A1
(43) Pub. Date: Apr. 9, 2026

(54) Detoxified Cholera Toxin as a Vaccine Adjuvant for Oral Vaccines

(71) Applicant: Inventprise, Inc., Redmond, WA (US)

(72) Inventor: Subhash V. Kapre, Redmond, WA (US)

(73) Assignee: Inventprise, Inc., Redmond, WA (US)

(21) Appl. No.: 19/352,224

(22) Filed: Oct. 7, 2025

Related U.S. Application Data
(60) Provisional application No. 63/704,157, filed on Oct. 7, 2024.

(51) Int. Cl. A61K 47/64

(52) U.S. Cl. CPC A61K 47/646

(57) ABSTRACT

The invention is directed to immunogenic compositions and vaccines, and methods for the manufacture and treatment and prevention of infections and, in particular, the preparation of immunogenic compositions and vaccines administered in conjunction with a detoxified cholera toxin as an adjuvant and a TLR agonist for oral administration, which may be encapsulated.

### REFERENCE TO RELATED APPLICATIONS

This application claims priority to U.S. Provisional Application No. 63/704,157, filed Oct. 7, 2024, the entirety of which is incorporated by reference.

### BACKGROUND

### 1. Field of the Invention

The present invention provides compounds, compositions, vaccines and methods for the treatment and prevention of viral infections and, in particular, immunogenic compositions and vaccines administered in conjunction with a detoxified cholera toxin as an adjuvant and a TLR agonist for oral administration, which may be encapsulated.

### 2. Description of the Background

A vaccine is a biological preparation that provides active acquired immunity to a particular infectious or even malignancy. The safety and effectiveness of vaccines has been widely verified. Vaccines contain an biological agent that resembles or mimics the disease-target causing microorganism. This agent is composed of weakened or killed forms of the microbe, its toxins, or one of its surface proteins or polysaccharides. Upon administration, the agent stimulates the body's immune system to recognize the target infection and amasses an immune response to eliminate that target which is maintained for a period of time as an immunological memory.

Vaccines can be prophylactic (preventing a future infection), or therapeutic (invoking the host's immune system to provoke an attack against the infection). To date, vaccinations are the safest and most effective method of preventing infectious diseases. In fact, vaccinations are responsible for the elimination of smallpox from the world and have severely reduced the incidences of diseases such as polio, measles, and tetanus from much of the world. The World Health Organization (WHO) reports that licensed vaccines are currently available for twenty-five different preventable infections.

There is overwhelming scientific consensus that vaccines are a very safe and effective way to fight and eradicate a great many infectious diseases. The immune system recognizes vaccine agents as foreign, destroys them, and retains the ability to remember that the agent is foreign, referred to as immune memory. When the foreign body is encountered once again, the host recognizes that body by immune memory and responds, by first neutralizing the foreign body before it can enter cells, and secondly by recognizing and destroying infected cells before that foreign body can multiply.

Once antibodies are produced, they may promote immunity in any of several ways, depending on the class of antibodies involved. Their success in clearing or inactivating a pathogen will depend on the amount of antibodies produced and on the extent to which those antibodies are effective at countering the strain of the pathogen involved, since different strains may be differently susceptible to a given immune reaction. In some cases vaccines may result in partial immune protection (in which immunity is less than 100% effective but still reduces risk of infection) or in temporary immune protection (in which immunity wanes over time) rather than full or permanent immunity. They can still raise the reinfection threshold for the population as a whole and make a substantial impact. They can also mitigate the severity of infection, resulting in a lower mortality rate, lower morbidity, faster recovery from illness, and a wide range of other effects. In addition, when a vaccinated individual does develop the disease vaccinated against, the disease is likely to be less virulent than in unvaccinated cases.

Vaccines led to the eradication of smallpox, one of the most contagious and deadly diseases in humans. Other diseases such as rubella, polio, measles, mumps, chickenpox, and typhoid are nowhere near as common as they were a hundred years ago thanks to widespread vaccination programs. As long as the vast majority of people are vaccinated, it is much more difficult for an outbreak of disease to occur, let alone spread. This effect is called herd immunity. Polio, which is transmitted only among humans, is targeted by an extensive eradication campaign that has seen endemic polio restricted to only parts of three countries (Afghanistan, Nigeria, and Pakistan). However, the difficulty of reaching all children, cultural misunderstandings, and disinformation have caused the anticipated eradication date to be missed several times.

Vaccines also help prevent the development of antibiotic resistance. For example, by greatly reducing the incidence of pneumonia caused by Streptococcus pneumoniae, vaccine programs have greatly reduced the prevalence of infections resistant to penicillin or other first-line antibiotics.

Many vaccines can be administered orally, although most are administered through intravenous (IV) injection, subcutaneous (SQ), intranasal (IN), intraperitoneal (IP) injection, trans dermally (TD) such as with microneedles, or intramuscular (IM) injection.

There are limitations on the effectiveness of vaccines. Protection can fail or be insufficient to provoke an immune response because of attenuation of the vaccine's active agent, failure to complete a vaccination regimen, or a poor route of administration. It is often the latter two reason why vaccines are not as effective as hoped in certain regions. Regions where difficulties arise in delivery of the vaccines, getting individuals to the health care professionals who will administer the vaccine, sometime repeatedly.

By way of example, a host does not develop antibodies after vaccination instantaneously. The body's innate immunity may be activated in as little as twelve hours, but adaptive immunity can take 1-2 weeks to fully develop, often requiring a booster vaccination. During that time, the host can still become infected. Thus, by missing the booster vaccination may all but destroy the positive protection that can be achieved.

Accordingly, there is a need to promote the ease of getting vaccines to individuals, the ease of administering the vaccines to individuals, and the any perceived or actual discomfort associated with administering vaccines.

### SUMMARY OF THE INVENTION

The present invention overcomes the problems and disadvantages associated with current strategies and designs and provides new vaccines, compositions and methods for the treatment of infections.

One embodiment of the invention is directed to immunogenic compositions and vaccines comprising detoxified cholera toxin (dCT) forming complexes. Complexes may be further coupled with a toll-like receptor agonist, and/or be encapsulated for delayed release of the immunologically active agent.

Another embodiment of the invention is directed to the manufacture of immunogenic composition as disclosed herein.

Another embodiment of the invention is directed to methods of treatment of infections and other detrimental medical conditions with the immunogenic compositions disclosed herein.

Other embodiments and advantages of the invention are set forth in part in the description, which follows, and in part, may be obvious from this description, or may be learned from the practice of the invention.

### DESCRIPTION OF THE INVENTION

Vaccines for the treatment and prevention of many different viral and bacterial infection are currently available. Many of these vaccines must be administered intra venous (IV) injection, subcutaneous (SQ), trans dermal (TD), intraperitoneal (IP) injection, intranasal (IN), or intramuscular (IM) injection as the antigenic components would otherwise be degraded in the oral and stomach cavities with a substantial loss of effectiveness due to thermal and/or pH instability.

It has been known for some time that cholera toxin (CT) possesses adjuvant activity when associated with vaccine compositions. Clinical trials in mice and other mammals showed that co-administration of a vaccine with CT would result in substantial increases in the levels of the immune response generated as compared to the vaccine alone. Of course, CT is toxic at most dosages and currently is used sparingly if at all. A number of methods have been developed to delete or overcome toxicity due to CT with some success. Cholera toxin (CT) can be detoxified mechanically, with autoclaving, chemically upon treatment with glutaraldehyde or sodium hypochlorite under defined conditions, or genetically by deletion of certain nucleic acid sequences.

It has been surprisingly discovered that detoxified cholera toxin (dCT) can be coupled with a vaccine and remain as an adjuvant when administered. Preferably, the dCT is conjugated to the antigenic components of the vaccine such as the polysaccharide or protein portions. In addition, the dCT-vaccine combination can also be coupled with a toll-like receptor (TLR) agonist to further boost the resulting immune response after administration. The dCT, vaccine, and TLR components are preferable conjugated together as a single complex for administration to a patient in need. dCT has the ability to activate immune cells and enhance antigen presentation in association with the particular antigen of the vaccine. Dendritic cells and macrophages are activated, increasing cytokine production and promoting a more robust immune response. This enhances both humoral and cellular immunity, especially at mucosal surfaces such as the gut. d CT binds to GM1 ganglioside receptors, which are widely present on mucosal surfaces. This assist the efficient delivery of the associated antigen to the immune system, making it an excellent mediator for antigen presentation. As CT is typically associated with mucosal (e.g., oral or nasal) vaccines, dCT is especially useful with oral vaccines, and also injectable. The strength of the immune response attributable to dCT as an adjuvant with oral and injectable vaccines improves antigen uptake and immune activation similar to standard injectable adjuvants, and especially for orally administered vaccines.

Vaccine components include antigens of the infectious agent such as, for example, polysaccharides, proteins, VLPs, or similar antigens. Preferably, this conjugated complex can be encapsulated with a coating agent that is soluble at pH levels above 7, and pulverized to bring the materials of the composition to 10 micron or less in size. The resulting composition is placed in a suspension as a vaccine having similar immunogenic properties as a injectable vaccine. This allows for the conversion of otherwise injectable vaccines to oral form.

Immunogenic composition and vaccines to which this disclosure applies include vaccines against any infectious agent including bacterial and viral infectious agents. Bacterial infectious agents include various polysaccharides (PS) and/or proteins of different species, subspecies and/or serotypes of Staphylococcus, Streptococcus, Salmonella, Pneumococcus, Campylobacter, Pseudomonas, Neisseria, Mycobacteria, Corynebacteria, Enterobacter, Vibrio, Bordetella, Clostridium, Yersinia, Listeria, Treponema, and many others. Viral infectious agents include various PS and/or proteins of different species, subspecies and/or serotypes of influenza virus (A, B, or C), coronavirus, hepatitis virus, papilloma virus, norovirus, herpes simplex virus, herpes zoster virus, human immunodeficiency virus, respiratory syncytial virus, Encephalitis virus, any of a the cold viruses, pox virus, measles virus, mumps virus, rotavirus, rubella virus, Hantavirus, and many others. Any vaccination of the commercially available vaccine can be formulated with the disclosures herein to be administered orally and with greater effectiveness.

As discussed, the vaccine components are mixed with a dCT. CT holotoxin are much too toxic for use in humans. To try and address the unfortunate toxicity problems of holotoxin, a non-toxic recombinant derivatives of CT. CTB was developed. This form of CT is a poor adjuvant but coupling the antigens to CTB increased the immunogenicity by increasing receptor-mediated uptake and presentation by the APCs. Preferably CT is detoxified recombinantly. CT contains a ctxA subunit, which is the A subunit of the V. cholerae toxin responsible, when functional, for many of the symptoms of cholerae (e.g., nausea, diarrhea etc.). dCT contains deletions of the entire core genetic element, which includes the ctxA/B, and a region known as the Intestinal Colonization Factor (ICF). Alternatively, CT can be treated with glutaraldehyde and/or sodium hypochlorite to be detoxified chemically, or exposed to high temperatures for long periods of time (e.g., autoclaved) to be detoxified mechanically.

The vaccine and dCT can be coupled, preferably with conjugation. Various conjugations method are disclosed in U.S. Pat. Nos. 11,027,022; 10,967,071; 10,576,144, and 7,166,708; all of which are incorporated by reference). Preferably the dCT and vaccine are coupled to TLR agonists, which are known to boost the resulting immune response. Preferably, coupling is through conjugation forming a dCT, vaccine, TLR agonist complex. Once the complex is formed, preferably the materials is encapsulated with a coating agent that is soluble at pH levels above 7, and/or pulverized to bring the materials of the composition to 10 micron or less in size. This encapsulated complex or simply encapsulate antigen prevent enzymatic digestion and pH degradation in the stomach, so that upon oral administration the vaccine moves to gut where it can mediate and enhance the immune response. Virus-like particles (VLPs) or similar antigens can also be complexed with dCT and TLR agonists could also be conjugated.

One embodiment of the invention is directed to immunogenic compositions and vaccines containing components include antigens of the infectious agent such as, for example, polysaccharides, proteins, VLPs, or similar antigens, and dCT. Preferably the dCT is conjugated to active components of the immunogenic compositions or vaccines. The dCT serves as an adjuvant, especially for orally administered immunogenic compositions and vaccines. The immunogenic compositions and vaccines coupled with dCT cab also be coupled with TLR agonists, which further stimulate the immune system.

Preferably, this conjugated complex can be encapsulated with a coating agent that is, at least preferably soluble at pH levels above about 6, above about 7, or above about 8, and/or to delay release of the immunologically active agent. Also preferably, the material components can be pulverized to bring the materials of the composition to a size of 10 o micron or less, 75 micron or less, 50 micron or less, 25 micron or less, 10 micron or less, or 5 micron or less. The resulting composition is preferably placed in a suspension as a vaccine having similar immunogenic properties as a injectable vaccine. This allows for the conversion of otherwise injectable vaccines to oral form.

Preferably, purified or isolated complexes can be freeze dried and formed into tablets or capsules or prepared as a paste or liquid composition. Compositions or complexes may contain pharmaceutically acceptable carries such as one or more of water, oils, alcohols, flavoring agents, surfactants, and other chemical components typical for vaccine compositions.

Preferably, complexes can be encapsulated with commercially available materials to delay release or exposure to the host immune system of the active components of the vaccine. Commercially available method for the encapsulation of immunogenic compositions and vaccines are described in U.S. Pat. Nos. 11,826,418; 11,628,208; 9,248,176; 9,114,174; and 5,730,989; all of which are specifically incorporated by reference).

Another and related embodiment of the invention is directed to methods for the manufacture of complexes of the disclosure. Preferably the immunogenic compositions and vaccines are prepared, first with the commercially available immunogenic compositions and vaccines. Next, CT is obtained and detoxified, preferably by recombinant technology forming deleted form of CT that are detoxified. The dCT is coupled to the composition, preferably by conjugation. Next, the composition coupled to dCT is couple with a TLR agonist, preferably by conjugation. The resulting complexes can be purified for administration. Complexes may be dry to be administered as tablets or capsules, or prepared as paste or liquid form to be swallowed or administered IN, or trans dermally though microneedle delivery.

Another and related embodiment of the invention is directed to methods for the administration of complexes of the disclosure. Administration is preferably oral, but may be injectable. Preferably administration does not require the technical services of health care professionals and vaccines can be taken orally or IN, in other words not requiring injection and the costs or materials and technical services associated with injectable vaccines.

Other embodiments and uses of the invention will be apparent to those skilled in the art from consideration of the specification and practice of the invention disclosed herein. All references cited herein, including all publications, and all U.S. and foreign patents and patent applications are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A complex comprising:
an immunological composition containing an immunologically active agent; and
a detoxified cholera toxin.

2. The complex of claim 1, wherein the immunologically active agent is conjugated to the detoxified cholera toxin.

3. The complex of claim 1, which is a tablet or capsule.

4. The complex of claim 1, which is a paste or liquid.

5. The complex of claim 1, further comprising a pharmaceutically acceptable carrier.

6. The complex of claim 1, further comprising a toll-like receptor agonist.

7. The complex of claim 1, wherein the toll-like receptor agonist is conjugated to the immunologically active agent and/or the detoxified cholera toxin.

8. The complex of claim 1, which is encapsulated.

9. A method for the manufacture of the complex of claim 1 comprising;
obtaining the immunological composition containing the immunologically active agent;
detoxifying cholera toxin;
conjugating the agent to the detoxified cholera toxin forming the complex; and
isolated the complex.

10. The method of claim 9, further comprising conjugating the complex with a toll-like receptor agonist.

11. The method of claim 9, further comprising encapsulating the isolated complex.

12. The method of claim 9, further comprising freeze drying the isolated complex.

13. The method of claim 9, further comprising adding a pharmaceutically acceptable carrier to the isolated complex.

14. A method for administering the complex of claim 1 to an individual comprising:
obtaining the complex;
administering the complex to an individual wherein administration is oral, intranasal or transdermal.

15. The method of claim 14, wherein the complex is a tablet or capsule and administration is oral.

16. The method of claim 14, wherein the complex is a paste, gel, or liquid and administration is transdermal via microneedles.
